(19) United States
(12) Patent Application Publication
(10) Pub. No.: US 2003/0147960 A1
(43) Pub. Date: Aug. 7, 2003

(54) Ionic antimicrobial coating

(76) Inventors: Tung-Liang Lin, Acton, MA (US); Min-Shyan Sheu, Chelmsford, MA (US)

Correspondence Address: FISH & RICHARDSON PC, 225 FRANKLIN ST, BOSTON, MA, 02110, US

(21) Appl. No.: 10/371,620

(22) Filed: Feb. 21, 2003

Related U.S. Application Data
(63) Continuation-in-part of application No. 09/829,691, filed on Apr. 10, 2001, now abandoned.

(51) Int. Cl. A61K 31/704; A61K 31/65; A61K 31/58; A61K 31/545; A61K 31/497; A61K 31/155; A61K 33/38

(52) U.S. Cl. 424/486; 424/618; 514/35; 514/200; 514/192; 514/152; 514/172; 514/470; 514/253.08; 514/254.07; 514/635

(57) ABSTRACT

This invention relates to an ionic antimicrobial coating. Such a coating may contain (1) a water-insoluble polymer having a first ionized group and (2) an antimicrobial agent having a second ionized group with a charge opposite to that of the first ionized group, in which the antimicrobial agent is attached to the water-insoluble polymer via an ionic bond between the first ionized group and the second ionized group.

### CROSS REFERENCE TO RELATED APPLICATION

This application is a continuation-in-part and claims the benefit of priority of U.S. application Ser. No. 09/829,691, filed on Apr. 10, 2001.

### BACKGROUND

A conventional antimicrobial coating is prepared by physically “entrapping” an antimicrobial agent in a polymer matrix. The antimicrobial agent is released by diffusion at a rate related to several factors, e.g., the solubility and size of the antimicrobial agent, and the pH of the medium in which the antimicrobial coating is placed.

In an aqueous medium, a conventional antimicrobial coating first releases the antimicrobial agent at a high rate and exhibits high antimicrobial activity. The release rate and antimicrobial activity then decrease over time. Thus, a conventional antimicrobial coating is generally effective in preventing microbe growth for only a short period of time.

### SUMMARY

The present invention relates to an antimicrobial coating on a device (e.g., a urethral stent or a heart valve) that can slowly release an antimicrobial agent to prevent microbe growth.

In one aspect, the invention features a coating that includes a water-insoluble polymer and an antimicrobial agent, each of which contains an ionized group. The two ionized groups, one on the polymer and the other on the antimicrobial agent, have opposite charges. The antimicrobial agent is linked to the water-insoluble polymer via an ionic bond between the two ionized groups. The water-insoluble polymer preferably contains a plurality of ionized groups, which may be different. The antimicrobial agent may also have more than one ionized group.

The water-insoluble polymer, which constitutes the matrix of the antimicrobial coating, can be an epoxy polymer, polyester, polyurethane, polyamide, polyacrylamide, poly(acrylic acid), or polyphosphazene, each containing ionized groups; or a copolymer thereof. The antimicrobial agent can be a biguanide salt, silver salt, polymyxin, tetracycline, aminoglycoside, penicillin, sulfadiazine, bacitracin, neomycin, miconazole, fusidic acid, nitrofurazone, norfloxacin, or cephalosporin, each containing one or more ionized groups. An antimicrobial coating can include two or more different water-insoluble polymers and two or more different antimicrobial agents.

The above-described antimicrobial coating optionally includes a hydrophilic polymer that is blended with the water-insoluble polymer. The hydrophilic polymer absorbs water to the coating and facilitates the release of the antimicrobial agent. A hydrophilic polymer is a polymer containing hydrophilic groups. Examples of such a hydrophilic polymer include poly(N-vinyl lactam), polyvinylpyrrolidone, polyethylene oxide, polypropylene oxide, cellulose, polyanhydrate, polyvinyl alcohols, polysaccharide, or polyvinyl ether, or a copolymer thereof.

In another aspect, the invention features a coating that includes a water-soluble polymer, a water-insoluble polymer, and an antimicrobial agent. Each of the water-soluble polymer and the antimicrobial agent contains an ionized group. The two ionized groups have opposite charges. The antimicrobial agent is linked to the water-soluble polymer via an ionic bond between the two ionized groups. The water-soluble polymer, which is hydrophilic in nature, facilitates the release of the antimicrobial agent. Examples of the water-soluble polymer include poly(N-vinyl lactam), polyvinylpyrrolidone, polyethylene oxide, polypropylene oxide, cellulose, polyanhydrate, polyvinyl alcohols, polysaccharide, or polyvinyl ether, or a copolymer thereof. The water-insoluble polymer serves as the matrix of the coating. For examples of the water-insoluble polymer and the antimicrobial agent, see above.

Each polymer component (i.e., the water-insoluble polymer, the water-soluble polymer, and the hydrophilic polymer) in the coating can be cross-linked with a cross-linking agent. When two different polymer components are present, they can also be cross-linked to each other. Examples of a suitable cross-linking compound include any organic compounds containing one functional group (such as a carbodiimide group), or containing two or more functional groups (such as aziridine groups, epoxy groups, silane groups, or isocyanate groups).

When an antimicrobial coating of this invention is placed in an aqueous medium, the antimicrobial agent, which is ionically bonded to the water-insoluble polymer or the water-soluble polymer, is slowly released via ion exchange. Consequently, effective concentrations of the released antimicrobial agent near the coating are maintained for a longer period of time, as compared with a conventional antimicrobial coating.

The details of one or more embodiments of the invention are set forth in the description below. Other features, objects, and advantages of the invention will be apparent from the description and drawings, and from the claims.

### DETAILED DESCRIPTION

The invention is based on a discovery that an antimicrobial agent can be ionically bonded to a coating polymer on a device so that it is released in a controlled manner.

An antimicrobial coating of this invention can be prepared, for example, by the following method: A water-insoluble polymer that contains ionic groups is first added to an aqueous solvent to form a solution (including a dispersion). Such a polymer solution can also be prepared by emulsion-polymerizing monomers in an aqueous solution. An antimicrobial agent that also contains ionic groups is then added to the solution. The pH of the solution is adjusted so that essentially all of the ionic groups with opposite charges on both the water-insoluble polymer and the antimicrobial agent are ionized. An antimicrobial coating solution is formed. A gentle stirring facilitates the interaction between the ionized groups on the water-insoluble polymer and the antimicrobial agent. The coating solution can then be applied to, and form an antimicrobial coating on, a surface of a substrate (e.g., an implantable medical device). For example, a substrate is dipped in the coating solution, removed from it, and then dried in air or in a heated oven. A substrate can also be coated by spray coating, spin coating, or paint coating. The coating thus obtained contains ionic bonding formed between the ionized groups on the water-insoluble polymer and the antimicrobial agent. The coating renders the substrate surface inhospitable to microorganisms and thereby prevents their colonization on it. The surface of the substrate, optionally, can be pretreated, e.g., with oxygen plasma, for better adhesion.

A suitable pH of the coating solution can be determined based on the pKa values of the respective ionic groups on the water-insoluble polymer and on the antimicrobial agent. For example, one can use poly(ethylene-co-acrylic acid) (PEA) as a water-insoluble polymer and AgCl as an antimicrobial agent. The pH of the solution can be adjusted to a value (e.g., 9) well above the pKa of the COOH group (4-5) on PEA so that essentially all of the COOH groups on PEA are ionized to form COO⁻ groups. Preferably, a weak base, such as ammonium hydroxide, is used to increase the pH of the solution. AgCl, when dissolved in an aqueous solution, is completely ionized. As a result, ionic bonding is formed between COO⁻ and Ag⁺ in the coating prepared from this solution.

An antimicrobial coating can also be prepared using a water-soluble polymer and an antimicrobial agent, each containing ionic groups. As an example, the water-soluble polymer and the antimicrobial agent, as well as a water-insoluble polymer, are added to an aqueous solvent to form a solution. The pH of the solution is adjusted so that essentially all of the ionic groups on both the water-insoluble polymer and the antimicrobial agent are ionized. An antimicrobial coating solution is formed after gentle stirring for an extended period of time. The coating solution can be applied to a substrate by the methods described above to form a coating. In use, such a coating does not substantially lose the water-soluble polymer to an aqueous environment due to presence of the water-insoluble polymer in the coating as a matrix.

The antimicrobial performance of a coating of this invention can be enhanced by including a hydrophilic polymer (e.g., a water-soluble polymer) and a cross-linking agent in the coating solution. For example, presence of a hydrophilic polymer facilitates the capture of water to create a semi-permanent water zone around the coating, which in turn helps to release the antimicrobial agent and prevent adhesion of microbes. A cross-linking agent stabilizes the polymer component(s) of the coating and prolongs the release of antimicrobial agents. As an example, one can use PEA as a water-insoluble polymer and a compound having two aziridine groups as a cross-linking agent to cross-link the PEA polymer. More specifically, two carboxyl groups on two PEA molecules can, respectively, react with the two aziridine groups on the cross-linking agent, resulting in formation of cross-linked PEA molecules. The release rate of an antimicrobial agent can be adjusted by using different types and amounts of the antimicrobial agent, the polymer component(s), and the cross-linking agent.

The effectiveness of an antimicrobial coating can be determined by conducting a “zone of inhibition” test. In this test, a substrate coated with an antimicrobial coating of this invention is inserted into a lawn of bacteria grown on an agar in such a way that the coating comes in contact with the bacteria. The antimicrobial agent released from the coating effectively inhibits microbe growth in a zone around the coated substrate. The zone, called “zone of inhibition,” is then measured. The size of the zone is an indicator of whether an effective amount of an antimicrobial agent is released from a coating. Conventional coatings release antimicrobial agents in amounts that dramatically decrease over time. In some cases, they become ineffective in only two days. In contrast, antimicrobial coatings disclosed herein, unexpectedly, release antimicrobial agents in effective amounts up to 60 days.

Without further elaboration, it is believed that one skilled in the art, based on the description herein, can utilize the present invention to its fullest extent. The following specific examples, which describe preparation and uses of several antimicrobial coatings of this invention, are therefore to be construed as merely illustrative, and not limitative of the remainder of the disclosure in any way whatsoever.

### EXAMPLE 1

A 15% aqueous poly(ethylene-co-acrylic acid) (PEA) dispersion was purchased from Mica Corporation (Stratsford, Conn.). The pH of this dispersion was 9.2. At this pH, essentially all COOH groups (pKa=4 to 5) on PEA are ionized to form COO⁻ groups. A 20% aqueous polyvinylpyrrolidone (PVP) solution was prepared by directly dissolving PVP into de-ionized water.

41.67 g of the PEA dispersion was first diluted with 19.58 g of de-ionized water. To the diluted PEA dispersion were sequentially added 37.50 g of the PVP solution and 1.00 g of silver chloride. The mixture thus obtained was gently stirred for at least 24 hours until the aqueous phase became saturated with silver chloride, and then filtered through a 50 mm filter to remove excess silver chloride. The filtrate was used as an antimicrobial coating dispersion.

High-density polyethylene (HDPE) 20 French tubes (0.263×0.229×12″) from Duall Plastics (Athol, Mass.) were treated with oxygen plasma at 100 mTorr and 300 watts for 2 minutes, primed with the 15% PEA dispersion, and heated at 60° C. for 40 minutes. The tubes were subsequently coated with the antimicrobial coating dispersion and heated at 60° C. overnight.

The coated tubes were tested in a 30-day release study. In this study, the coated tubes were soaked in artificial urine and collected at five-day intervals. Each of the collected tube was then subjected to an inhibition zone test. See Sawan et al. (Eds) Antimicrobial/Anti-Infective Materials, Chapter 13, 2000, Technomic Publishing Company, Inc., Lancaster, Pa., which is herein incorporated by reference. More specifically, it was vertically inserted into a lawn of Staphylococcus epidermidis grown on an agar for 24 hours in such a way that the coating came in contact with the bacteria. The results show that the sizes of the inhibition zone were unexpectedly the same (2.6 mm) throughout the entire study period.

### EXAMPLE 2

41.67 g of the PEA dispersion described in Example 1 was diluted with 19.58 g of de-ionized water. To the diluted PEA dispersion were sequentially added 37.50 g of the PVP solution also described in Example 1 and 1.00 g of silver chloride. 1.25 g of a cross-linking agent containing two or more aziridine groups (CX-100, NeoResins, Wilmington, Mass.) was added after gentle stirring the above dispersion for 24 hours and filtering the dispersion. The dispersion thus obtained was further stirred for 30 minutes, resulting in an antimicrobial coating dispersion.

HDPE 20 French tubes were pretreated with oxygen plasma at 100 mTorr and 300 watts for 2 minutes, primed with the acrylic polymer dispersion, heated at 60° C. for 40 minutes, coated with the coating dispersion, and heated again at 60° C. overnight.

The coated tubes were tested in a 30-day release study and following the procedure described in Example 1. The results show that the sizes of the inhibition zones were the same (2.0 mm) throughout the entire study period.

### EXAMPLE 3

A 38% aqueous polyurethane dispersion (NeoRez R-9621) was purchased from NeoResins, Inc (Wilmington, Mass.). The polyurethane in this solution contains COOH groups. The pH of this dispersion was adjusted to 8.0 by the supplier with triethylamine. At this pH, essentially all COOH groups (pKa=4 to 5) on polyurethane are ionized to form COO— groups.

A priming solution was prepared by mixing 200.00 g of the 38% aqueous polyurethane solution, 80.00 g of de-ionized water, and 3.00 g of the cross-linking agent described in Example 2.

A coating dispersion was prepared by the following procedure: 25.00 g of the 38% aqueous polyurethane dispersion was first diluted with 25.00 g of de-ionized water. To the diluted polyurethane dispersion were sequentially added 13.75 g of the 20% PVP solution described in Example 1 and 0.52 g of silver chloride. The mixture thus obtained was gently stirred for at least 24 hours until the dispersion became saturated with silver chloride, and filtered through a 50 mm filter to remove excess silver chloride. 0.50 g of the cross-linking agent was then added to the filtrate. The dispersion thus obtained was stirred for another 30 minutes, resulting in an antimicrobial coating dispersion.

Three more coating dispersions were prepared by following the same procedure, except that 0.55 g, 0.575 g, and 0.625 g of the cross-linking agent were respectively used.

HDPE 20 French tubes were pretreated with oxygen plasma at 250 mTorr and 250 watts for 2 minutes. The pretreated tubes were subsequently primed with the above-described priming dispersion, heated at 60° C. for 40 minutes, coated with the four coating dispersions, respectively, and heated again at 60° C. overnight.

The coated tubes were tested in a 30-day release study and following the procedure described in Example 1. The results show that the sizes of the inhibition zones of these four coatings were the same (1.85 mm) throughout the entire study period.

### EXAMPLE 4

An antimicrobial coating dispersion of a different composition was prepared by following the procedure described in Example 3. The dispersion included 50.0 g of the 38% polyurethane dispersion, 50.0 g of the 20% PVP solution, 60.0 g of de-ionized water, 0.6 g of silver chloride, and 1.0 g of the cross-linking agent.

HDPE 20 French tubes were pretreated with oxygen plasma at 100 mTorr and 300 watts for 4 minutes. The tubes were primed with a priming dispersion including 140.0 g of the 38% polyurethane dispersion, 56.0 g of de-ionized water, and 2.1 g of the cross-linking agent, and heated at 65° C. for 30 minutes. The primed tubes were then coated with the antimicrobial coating described above, and heated again at 65° C. for 3 hours.

The coated tubes were tested in a 60-day release study and following the procedure described in Example 1. They were collected at five-day intervals and then used in a zone of inhibition test against staphylococcus epidermidis and Escherichia coli. The results show that the size of inhibition zone remained constant for 50 days (3.0 mm) against Staphylococcus epidermidis and for 60 days (2.0 mm) against Escherichia coli throughout the entire study period.

### Example 5

An 8% AgCl solution was prepared by dissolving 2.4 g of AgCl in 27.6 g of 28% ammonia hydroxide aqueous solution.

Antimicrobial discs were prepared by the following procedures: 2.5 g of 38% aqueous polyurethane dispersion (NeoRez R-9621) was diluted with 2.2 g of de-ionized water. To the diluted polyurethane dispersion were sequentially added 1.5 g of the 20% PVP solution described in Example 1 and 0.313 g of the 8% AgCl solution prepared above. The mixture thus obtained was gently stirred for 30 minutes, followed by addition of 0.05 g of the cross-linking agent described in Example 2. An antimicrobial coating dispersion was obtained by gently stirring for another 30 minutes. 15 mL of this antimicrobial coating dispersion was poured into a glass petri dish and dried in a 65° C. oven overnight. Antimicrobial discs were obtained by removing the dried membrane from the petri dish with a puncher having an inner diameter of 0.25 inch. The average weight of each antimicrobial disc was 0.0139 g. The average weight of silver ion in each antimicrobial disc was 278 μg.

The antimicrobial discs were placed in three different solutions of different ion concentrations: (1) de-ionized water, (2) 53 mM citric buffer solution containing 0.9% NaCl, and (3) 530 mM citric buffer solution containing 9% NaCl. The release of Ag⁺ ion from the antimicrobial discs into the solutions was monitored daily for two weeks using a UV-visible spectroscopy (Spectronic Genesys 5, Milton Roy, Inc., Buffalo, N.Y.) at 636 nm. The Ag⁺ concentrations were determined based on a Ag⁺ standard solution.

The antimicrobial discs showed an increase in the release rate of Ag⁺ with the increase of the Na⁺ concentration. The release rate was the slowest when the discs were placed in de-ionized water. These results indicate that the release of Ag⁺ is via ion exchange. In other words, ionic bonding was formed between Ag⁺ and the COO⁻ groups on polyurethane in the antimicrobial discs.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. An antimicrobial coating comprising
a water-insoluble polymer having a first ionized group, and
an antimicrobial agent having a second ionized group with a charge opposite to that of the first ionized group;
wherein the antimicrobial agent is attached to the water-insoluble polymer via an ionic bond between the first ionized group and the second ionized group.

2. The antimicrobial coating of claim 1, further comprising a hydrophilic polymer that is blended with the water-insoluble polymer.

3. The antimicrobial coating of claim 2, wherein the water-insoluble polymer is an epoxy polymer, polyester, polyurethane, polyamide, polyacrylamide, poly(acrylic acid), or polyphosphazene, or a copolymer thereof.

4. The antimicrobial coating of claim 2, wherein the antimicrobial agent is biguanide salt, silver salt, polymyxin, tetracycline, aminoglycoside, penicillin, sulfadiazine, bacitracin, neomycin, miconazole, fusidic acid, nitrofurazone, norfloxacin, or cephalosporin.

5. The antimicrobial coating of claim 2, wherein the hydrophilic polymer is poly(N-vinyl lactam), polyvinylpyrrolidone, polyethylene oxide, polypropylene oxide, cellulose, polyanhydrate, polyvinyl alcohols, polysaccharide, or polyvinyl ether, or a copolymer thereof.

6. The antimicrobial coating of claim 3, wherein the antimicrobial agent is biguanide salt, silver salt, polymyxin, tetracycline, aminoglycoside, penicillin, sulfadiazine, bacitracin, neomycin, miconazole, fusidic acid, nitrofurazone, norfloxacin, or cephalosporin.

7. The antimicrobial coating of claim 4, wherein the hydrophilic polymer is poly(N-vinyl lactam), polyvinylpyrrolidone, polyethylene oxide, polypropylene oxide, cellulose, polyanhydrate, polyvinyl alcohols, polysaccharide, or polyvinyl ether, or a copolymer thereof.

8. The antimicrobial coating of claim 6, wherein the hydrophilic polymer is poly(N-vinyl lactam), polyvinylpyrrolidone, polyethylene oxide, polypropylene oxide, cellulose, polyanhydrate, polyvinyl alcohols, polysaccharide, or polyvinyl ether, or a copolymer thereof.

9. The antimicrobial coating of claim 7, wherein the water-insoluble polymer or the hydrophilic polymer is cross-linked with a cross-linking agent.

10. The antimicrobial coating of claim 8, wherein the water-insoluble polymer or the hydrophilic polymer is cross-linked with a cross-linking agent.

11. The antimicrobial coating of claim 9, wherein the water-insoluble polymer is poly(ethylene-co-acrylic acid), the hydrophilic polymer is polyvinylpyrrolidone, and the antimicrobial agent is silver chloride.

12. The antimicrobial coating of claim 10, wherein the water-insoluble polymer is poly(ethylene-co-acrylic acid), the hydrophilic polymer is polyvinylpyrrolidone, the antimicrobial agent is silver chloride, and the cross-linking agent is aziridine.

13. The antimicrobial coating of claim 10, wherein the water-insoluble polymer is polyurethane, the hydrophilic polymer is polyvinylpyrrolidone, the antimicrobial agent is silver chloride, and the cross-linking agent is aziridine.

14. An antimicrobial coating comprising
a water-soluble polymer having a first ionized group,
a water-insoluble polymer being blended with the water-soluble polymer, and
an antimicrobial agent having a second ionized group with a charge opposite to that of the first ionized group;
wherein the antimicrobial agent is attached to the water-soluble polymer via an ionic bond between the first ionized group and the second ionized group.

15. The antimicrobial coating of claim 14, wherein the water-soluble polymer is poly(N-vinyl lactam), polyvinylpyrrolidone, polyethylene oxide, polypropylene oxide, cellulose, polyanhydrate, polyvinyl alcohols, polysaccharide, or polyvinyl ether, or a copolymer thereof.

16. The antimicrobial coating of claim 14, wherein the water-insoluble polymer is an epoxy polymer, polyester, polyurethane, polyamide, polyacrylamide, poly(acrylic acid), or polyphosphazene, or a copolymer thereof.

17. The antimicrobial coating of claim 14, wherein the antimicrobial agent is biguanide salt, silver salt, polymyxin, tetracycline, aminoglycoside, penicillin, sulfadiazine, bacitracin, neomycin, miconazole, fusidic acid, nitrofurazone, norfloxacin, or cephalosporin.

18. The antimicrobial coating of claim 15, wherein the water-insoluble polymer is an epoxy polymer, polyester, polyurethane, polyamide, polyacrylamide, poly(acrylic acid), or polyphosphazene, or a copolymer thereof.

19. The antimicrobial coating of claim 15, wherein the antimicrobial agent is biguanide salt, silver salt, polymyxin, tetracycline, aminoglycoside, penicillin, sulfadiazine, bacitracin, neomycin, miconazole, fusidic acid, nitrofurazone, norfloxacin, or cephalosporin.

20. The antimicrobial coating of claim 16, wherein the antimicrobial agent is biguanide salt, silver salt, polymyxin, tetracycline, aminoglycoside, penicillin, sulfadiazine, bacitracin, neomycin, miconazole, fusidic acid, nitrofurazone, norfloxacin, or cephalosporin.

21. The antimicrobial coating of claim 17, wherein the water-soluble or the water-insoluble polymer is cross-linked with a cross-linking agent.

22. The antimicrobial coating of claim 18, wherein the antimicrobial agent is biguanide salt, silver salt, polymyxin, tetracycline, aminoglycoside, penicillin, sulfadiazine, bacitracin, neomycin, miconazole, fusidic acid, nitrofurazone, norfloxacin, or cephalosporin.

23. The antimicrobial coating of claim 19, wherein the water-soluble or the water-insoluble polymer is cross-linked with a cross-linking agent.

24. The antimicrobial coating of claim 20, wherein the water-soluble or the water-insoluble polymer is cross-linked with a cross-linking agent.

25. The antimicrobial coating of claim 22, wherein the water-soluble or the water-insoluble polymer is cross-linked with a cross-linking agent.
